(19) United States
(12) Patent Application Publication
(10) Pub. No.: US 2015/0141414 A1
(43) Pub. Date: May 21, 2015

(54) Formulation of Stable Recombinant Alpha-Fetoprotein Conjugated with Anti-Tumor Substance in Target-Delivery System for Treatment of Cancer and Autoimmune Disease

(71) Applicant: Nana Legrand, Freehold, NJ (US)

(72) Inventor: Nana Legrand, Freehold, NJ (US)

(73) Assignee: LeGrand International LLC, Freehold, NJ (US)

(21) Appl. No.: 14/455,249

(22) Filed: Aug. 8, 2014

Related U.S. Application Data
(60) Provisional application No. 61/864,830, filed on Aug. 12, 2013.

(51) Int. Cl. A61K 47/48; A61K 47/34; C07K 14/47; A61K 31/538

(52) U.S. Cl. CPC A61K 47/48246; A61K 31/538; A61K 47/34; C07K 14/4715
USPC 514/229.8; 435/69.1

(57) ABSTRACT

Disclosed is a stable formulation based on rAFP conjugated with drug delivery system of various classes of pharmacological agents such as dactinomycin, cardionolide, and bufadinolide, among others. In another embodiment, the invention discloses a method of producing rAFP in a strain of methylotrophic Pichia pastoris yeast, allowing the yeast to replicate, and extracting the newly created rAFP. The method provides a nonpyrogenic, highly stable, recombinant, fully refolded rAFP and its fragments that are free from stable folding intermediates caused by non-native disulfide bridges and irreversible aggregates. The newly created rAFP is purified using a two-step chromatographic purification process in the presence of 0.5% polysorbate-20 and 10 mM inosine that prevents aggregation of the monomers of the target protein, increasing the yield of biologically active rAFP. The invented formulation of rAFP is conjugated with active pharmacological substances or target drug delivery system for prevention or treatment of oncological and autoimmune-diseases.

### CROSS REFERENCE TO RELATED APPLICATION

This application claims the benefit of U.S. Provisional Application No. 61/864,830 filed on Aug. 12, 2013. The above identified patent application is herein incorporated by reference in its entirety to provide continuity of disclosure.

### BACKGROUND OF THE INVENTION

### Field of the Invention

The present invention relates to the composition and method for obtaining stabilized and improved recombinant alpha-fetoprotein (rAFP) and to the method of utilizing conjugates of rAFP, or its fragments (domain thereof), for the effective therapy of cancer and autoimmune diseases. More particularly, the present invention pertains to compositions and method of producing a high yield of rAFP by the introducing rAFP into a strain of yeast and subsequently extracting the newly created rAFP, and to a method of purification through effective single or multistage purification processes before including it into complex pharmaceutical composition with various anti-cancer substances. Thus, the present invention also pertains to a method of combining the newly created rAFP with anti-cancer drugs for cancer therapy and treatment of autoimmune diseases.

One of the greatest challenges in cancer drug therapy is to maximize the effectiveness of the active agent while reducing its systemic adverse effects. Many widely-used chemotherapeutic agents present unfavorable physicochemical properties (e.g. low solubility, lack of chemical or biological stability) that hamper or limit their therapeutic applications. All these issues may be overcome by designing adequate drug delivery systems, such as nano-carriers that are particularly suitable for this purpose. Nano-systems can be used for targeted-drug release, treatment, diagnostic imaging and therapy monitoring, as the primary consideration is to control the drug concentration in the therapeutic window.

Cancer is a broad group of diseases that affect people of all ages. Cancer is characterized by unregulated cell growth in various parts of the body. Cancer cells divide and grow uncontrollably, forming malignant tumors, which may invade nearby parts of the body, as well as more distant parts of the body through the lymphatic system or blood stream.

Many treatment options for cancer exist with the primary ones including surgery, chemotherapy, radiation therapy, and palliative care. However, scientists and medical experts continue to search for improved methods of treating cancer and autoimmune diseases. In recent years, there has been an increasing interest in the use of alpha-fetoprotein (AFP) for the treatment of cancer because AFP has a unique ability to interact only with the pathological cells. Additionally, AFP receptors are only expressed in most tumor cells, and thus AFP could be used for targeting drugs to tumor cells. Thus, allowing for possibility of enhancing selectively delivery of anti-tumor drug in the treatment of cancer. AFP is normally synthesized in the liver, intestinal tract, and yolk sac of the fetus. In vitro and in vivo experiments have shown that AFP has both cell growth-stimulatory and -inhibitory activities, depending upon the target cell, the relative concentration of AFP, and the presence of other cytokines and growth factors. For example, AFP can inhibit the growth of many types of tumor cells, and in particular, inhibits estrogen-stimulated cell growth. Conversely, AFP stimulates the growth of normal embryonal fibroblasts. AFP has also been shown to have both immunosuppressive and immunoproliferative effects.

Current formulations for treating cancer and autoimmune disease involve manufactured pharmaceutical grade rAFP and its fragments (domains thereof), which can be used in medicines in form of conjugates with biodegradable approve for medicine polymers carrying the antineoplastic drugs (e.g., dactinomycin), cardiotonic steroids (e.g., oleadrin, digoxin, digitoxin), drug inhibitors of tyrosine and other kinases, inhibitors of Hedgehog and mammalian target of rapamycin (mTOR) signaling pathways, and anti-rheumatic substances (e.g., methotrexate, cyclophosphamide, sulfasalazine), among others.

Antineoplastic substances, and Dactinomycin (DAC), in particular, is widely used as an anticancer agent for treating a variety of tumors. Dactinomycin, also known generically as Actinomycin D, is a cyclic polypeptide-containing antibiotic that binds to DNA and blocks transcription. It binds to premelted double stranded DNA in transcription bubbles, and once bound, it is slow to dissociate. DNA in transcriptionally hyperactive cancer cells efficiently and rapidly binds DAC at low concentration (10 nM), which interferes with RNA synthesis and ultimately leads to cell death. Thus, DAC is an effective anti-cancer agent for treating tumors. For example, Cosmegen® (dactinomycin for injection) is used in combination with chemotherapy and/or a multi-modality treatment regimen to treat Wilms' tumor, childhood rhabdomyosarcoma, and Ewing's sarcoma, among others.

Cardiotonic steroids, cardinolide and bufadinolide, have been shown to have anticancer activities during various stages of carcinogenesis. These activities include antiproliferative effect via their regulation of the cell cycle, pro-apoptotic, and chemotherapy sensitization actions. Particularly, bufalin, is able to induce arrest in human malignant melanoma cells in the G2/M phase of the cell cycle. In lung cancer cells, bufalin up-regulates p21 WAF1 and suppresses cyclin D expression in response to the activation of p53, which are responsible for cell cycle progression from G1 to S phase, these changes prevent cells from entering the next phase of the cycle. Cardinolide and digitoxin cause cell cycle arrest in G2/M in a dose-dependent manner, resulting in a large increase in the number of cells in the sub-G0 phase. An antiproliferative effect of ouabain against human breast and prostate cancer cells mediates the depletion of the Na+/K+-ATPase through endocytosis and a degradation-dependent pathway, which in turn elevates the level of the cell cycle inhibitor p21. Inhibitors of tyrosine kinase, such as Erlotinib, Lapatinib, Vandetanib (inhibitors of tyrosine kinase receptors) or crizotinib, imatinib or nilotinib (non-receptor tyrosine kinase inhibitors) most of all are small and hydrophobic compounds, thus can rapidly reach their specific intracellular targets and inhibit the activation of related tyrosine kinases.

However, efficacy of DAC is restricted by dose limiting toxicity, multi-drug resistance (MDR), and several drug associated side effects. For instance, DAC is lethal to mice and rats at intravenous doses of 700 mcg/kg and 500 mcg/kg, wherein these amounts are 3.8 and 5.4 times the maximum recommended daily human dose on a body surface area basis, respectively. The oral LD50 of dactinomycin is 7.8 mg/kg and 7.2 mg/kg for a mouse and rat, respectively. Thus, DAC drug is highly toxic in both powder and solution form, and must be administered so that it is released into the body in a controlled manner.

The half-lethal dose (LD50) for cardiotonic steroids, particularly for bufalin under intravenous administration is: 740 mcg/kg for a mouse; and 140 mcg/kg for a cat. LD50 for digitoxin under intravenous injection is: 320 μg/kg for a pigeon; 730 μg/kg for a frog; and 4.1 mg/kg for a mouse. The half-lethal dose for tyrosine kinase inhibitors also is very high, particularly for vandetanib LD50 under intravenous administration in rats is 17-41 mg/kg body weight (recommended daily dose is 300 mg/kg in human); and for anti-rheumatic substances such as methotrexate, for example, LD50 under intravenous administration in rats is 14 mg/kg body weight.

In one embodiment, the present invention creates a stable pharmaceutical formulation based on rAFP, which maintains the quality of the drug produced using rAFP or its peptides. The present method utilizes simple operation and conserves time and energy to produce a high yield of rAFP with a higher purity to include in the pharmaceutical formulation with active anti-tumor substances previously incorporated in biodegradable polymeric nanoparticles. Particularly, antineoplastic substance, DAC, which previously was encapsulated in PLGA (PLGA-COOH), in a controlled release manner and then conjugated with rAFP, exhibit significant reducing the toxic effects against normal, healthy cells, while increasing its therapeutic activity and facilitate control of drug uptake. Encapsulation of DAC through rAFP conjugation results in more cytotoxicity against tumor cell lines compared with free DAC and unconjugated PLGA particles of DAC, increasing circulation time of the active dose before reaching the target cell. Furthermore, the use of nanoparticles based on biodegradable, biocompatible, and FDA approved components, such as PLGA, is advantageous in that its use facilitates their future transition into clinical trials.

In another embodiment, the method generally involves expressing of successfully heterogonous AFP into a strain of Pichia pastoris metylotrophic yeast (GS115 with phenotype His⁻, Mut+), using DNA recombinant technology and construction recombinant plasmid, electroporation and transferring technology and unique methods of extracting the newly created rAFP. The method provides a nonpyrogenic, highly stable, recombinant, fully refolded rAFP and its fragments that are free from stable folding intermediates caused from non-native disulfide bridges and irreversible aggregates. The primary advantage of the present invention is not only its stable composition, but more specifically its process of purification, which help prolong the shelf life of rAFP and eliminates the problems associated with endobacterial insemination of finished drug dosage forms. In this way, the present invention provides a pharmaceutical grade product that is safe, pure, and effective.

In another embodiment, the method generally involves encapsulated into a biocompatible/biodegradable polymeric matrix to achieve prolonged exposure, which was reach by the include the poly(lactide-co-glycolide) (PLGA) copolymer as a carrier. DAC, a cytostatic or other anti-tumor substances with very low water solubility was encapsulated in PLGA nanoparticles produced by a modified emulsification evaporation method and was conjugated with rAFP molecules through a carbodiimide conjugation reaction. These newly developed fully biodegradable rAFP coated PLGA nanoparticles represent a promising nanoparticulate drug delivery system for DAC and other anticancer agents. Using a PLGA carriers for anti-tumor substances give possibility to significantly decrease systemic toxic effects of effective anticancer agent and realize a selective delivery of medicine into the tumour tissue.

### DESCRIPTION OF THE PRIOR ART

Formulation of conjugated of highly purified rAFP producing in the strain of Pishia pastoris yeast and some pharmaceutical active anti-tumor substances previously incorporated in PLGA-COON have been disclosed in the prior art. These include compositions and methods that have been patented and published in patent application publications. Some of these patents describe human AFP that is produced from prokaryotic cells. Other patents describe a method that comprises the steps of using a cell transduced with a transgene that includes a nucleic acid molecule encoding AFP, a promoter, and a leader sequence. The transduced cell is then grown until the cell expresses AFP and the AFP is extracted. These methods, however, do not provide a high yield of pure AFP. The following is a list of prior art compositions and methods deemed most relevant to the present disclosure, which are herein described for the purposes of highlighting and differentiating the unique aspects of the present invention, and further highlighting the drawbacks existing in the prior art.

For example, U.S. Pat. No. 5,384,250 to Murgita (Murgita '250) discloses a method of producing human AFP in prokaryotic cells. The method includes providing a transformed prokaryotic cell including a recombinant DNA molecule encoding human AFP operably linked to sequence elements that are capable of directing expression of human AFP, and permitting the transformed cell to express human AFP. Similarly, U.S. Pat. No. 6,331,611 to Murgita (Murgita '611) discloses a substantially pure recombinant human AFP produced using prokaryote and insect cells. Murgita '611 discloses that the AFP is produced by culturing a transformed insect cell and recovering the biologically active human AFP or fragment or analog thereof.

U.S. Pat. No. 6,288,034 to Murgita (Murgita '034) discloses a method of using recombinant human AFP as an immunosuppressive agent, and cross-references to Murgita '250 for methods for producing such AFP. Similarly, U.S. Pat. No. 6,416,734 to Murgita (Murgita '734) discloses a method of inhibiting a neoplasm in a mammal by administering a therapeutically effective amount of recombinant human AFP. The method is based on using the human AFP made in a prokaryote as disclosed in Murgita '250 and Murgita '611.

The foregoing compositions and methods are directed toward producing human AFP in prokaryotic cells. While the foregoing references disclose that human AFP may be used in therapeutic compositions, the method of producing human AFP using prokaryote cells is limited in the fact that expression of AFP in prokaryotic systems typically produces misfolded and inactive protein that is aggregated and does not have the correct internal disulfide bonds. This misfolded AFP must be purified and refolded under conditions that allow for the formation of correct disulfide bonds, a difficult and time-consuming process, which results in a very low overall yield of active, useful protein. In contrast, the present method provides a nonpyrogenic, highly stable, recombinant, fully refolded rAFP and its fragments that are free from stable folding intermediates caused from non-native disulfide bridges and irreversible aggregates.

U.S. Pat. No. 7,208,576 to Mulroy discloses non-glycosylated human alpha-fetoprotein (ng.HuAFP), methods of production in transgenic animals and plants, and uses thereof. Generally, Mulroy discloses a method of producing ng.HuAFP and treating immunologic disorder such as cancer with ng.HuAFP. More specifically, Mulfoy discloses that the method of producing ng.HuAFP includes the steps of providing a cell transduced with a transgene that comprises a nucleic acid molecule encoding ng.HuAFP, a promoter, and a leader sequence, then growing the transduced cell such that the cell expresses and secretes ng.HuAFP. Alternatively, the method of producing ng.HuAFP includes the steps of providing a transgenic organism that includes a transgene having a nucleic acid molecule encoding ng.HuAFP, a promotor, and a leader sequence, then collecting the biological fluid that includes ng.HuAFP from the transgenic organism. Following purification from the biological fluid, ng.HuAFP is at least 80% pure, preferably 90% pure, more preferably 95% pure, and most preferably 99%. Accordingly, the methods of production of ng.HuAFP disclosed in Mulroy does not involve introducing rAFP into a strain of Pichia pastoris yeast, allowing the yeast to replicate, and extracting the newly created rAFP. Additionally, the impurity content of rAFP produced by the present method does not exceed 0.5%.

U.S. Published Patent Application Number 2012/0315697 to Pettit discloses a cell culture media containing combinations of proteins and methods of making the cell culture media, and methods of using the cell culture media to improve growth characteristics of cultured cells. In one embodiment, Pettit discloses a cell culture media including a cell culture media base and two or more proteins including alpha-fetoprotein. Pettit, however, does not disclose a method of producing rAFP and uses thereof for treating cancer. The present invention provides a stable pharmaceutical preparation based on rAFP or its fragments in complexes with various substances incorporated in certain drug delivery systems for targeting cancer cells.

U.S. Published Patent Application Number 2007/0253973 to Rosen relates generally to fusion proteins comprising AFP and methods of producing the same. In a preferred embodiment, the fusion proteins comprise at least one therapeutic protein or vaccine antigen and AFP. The therapeutic protein or vaccine antigen may comprise a peptide, antibody, fragment thereof, or variant thereof. The therapeutic protein or vaccine antigen is fused with AFP, an AFP fragment, or an AFP variant. The fusion protein is produced by culturing a host cell comprising a nucleic acid molecule having a polynucleotide. The purpose and intent of the method and composition of fusion proteins disclosed in Rosen, however, differ from the present invention. The present invention does not combine one or more whole peptides to create a new protein. Instead, the present method provides a high yield of a stable and highly purified form of rAFP that is suitable for treatment of cancer.

U.S. Pat. No. 7,910,327 to Benevolensky discloses a composition of rAFP and method of preparing the same. In order to obtain a high yield of the secreted protein with the required activity from a host cell, several additional genes are added to the plasmid encoding the AFP gene, the additional genes providing a high level of gene transcription, folding of the proteins in the process of secretion and the correct formation of disulfide bonds. In contrast, the present method does not require additional genes. Instead, the present method comprises a two-step chromatographic purification of rAFP in the presence of 0.5% Tween-20 and 10 mM inosine that prevents aggregation of the monomers of the target protein, thereby increasing the yield of biologically active rAFP.

U.S. Published Patent Application Number 2013/0131317 to Lindsay discloses a method of expressing secreted recombinant human AFP in the milk or urine of transgenic mammals. The method of producing AFP includes the steps of providing a cell transfected with a transgene that comprises a nucleic acid sequence encoding AFP, a milk-specific or a urine-specific promotor, and a leader sequence. Thereafter, the cell is grown to produce a mammal comprising milk-producing or urine-producing cells that expresses and secrete AFP into the milk or urine. Then, the milk or urine containing AFP is collected and AFP is purified. In contrast, the present method does not utilize the milk or urine of transgenic mammals. The present method of production of rAFP disclosed comprises introducing rAFP into a strain of Pichia pastoris yeast, allowing the yeast to replicate, and extracting the newly created rAFP.

U.S. Published Patent Application Number 2013/0143313 to Niazi discloses a harvesting device for capturing a biological product directly by binding the secreted biological product with a resin, discarding the nutrient medium, and eluting the biological product as a concentrated solution. In this way, the device of Niazi allows a user to combine the steps of recombinant expression and separation of a biological product. While the present method involves the step of conducting elution of the target protein, the specific device that is used to conduct the elution is not of primary relevance with regard to the intent of the present invention, which portends to provide a stable pharmaceutical preparation based on rAFP or its fragments in complexes incorporated in drug delivery systems for treating cancer.

U.S. Pat. No. 5,206,153 to Morinaga discloses a method of producing recombinant human AFP in which a DNA encoding a signal peptide for a rat AFP is fused with a DNA encoding a human AFP. The method comprises the steps of fusing a DNA sequence coding a signal peptide for a rat AFP into a terminal site of cDNA for human AFP to form a chimera DNA, inserting the chimera DNA into a vector, inserting the vector into a yeast cell, culturing the yeast cell in a culture medium, and isolating human AFP from the cultured medium. The average yield of the Morinaga method is approximately 62.75 μg from one liter of the culture medium. In contrast, the average yield of the present method is approximately 7 to 9 mg from one liter of the culture medium. Accordingly, the present invention significantly increases the production of rAFP.

Finally, U.S. Pat. No. 7,662,584 to Penttila discloses methods for increasing the amount of protein secreted by recombinant eukaryotic cells. In one embodiment, the method comprises the steps of inducing an unfolded protein response (UPR) by increasing the presence of a HAC1 protein in a fungal cell. The presence of the HAC1 protein be increased by a number of methods known in the art. For instance, the HAC1 gene can be overexpressed by using vectors and promoters. The HAC1 may be increased in a cell by transformation of the fungal cell by a nucleic acid comprising a UPR inducing form of a HAC1 recombinant nucleic acid. The purpose and intent of the method of Penttila, however, differ from the present invention. Penttila provides a method to increase the amount of any protein secreted by recombinant eukaryotic cells. In contrast, the present invention provides a method of increasing the yield of stable and biologically active rAFP.

These prior art compositions and methods have several known drawbacks. The present invention discloses a method of increasing the production and purification of rAFP. The present method includes consecutive use of the ultrafiltration steps and precipitation of the target protein with ammonium sulfate. This results in significant reduction in the treated culture medium volume at the first stages of purification, which is important for scaling the manufacturing process of rAFP production. The present method also discloses a two-step chromatographic purification of rAFP in the presence of 0.5% Tween-20 and 10 mM inosine that prevents aggregation of the monomers of the target protein, thereby increasing the yield of biologically active rAFP. The biologically active rAFP can be used immediately after dissolution for parenteral administration. Furthermore, the present invention provides a method of conjugating DAC loaded PLGA to rAFP to decrease the systematic toxicity of DAC and to increase its therapeutic benefits. In this way, the present invention overcomes the challenge of encapsulating DAC into polymeric nanoparticles.

It is therefore submitted that the present invention substantially diverges in design elements from the prior art, which overcomes the disadvantages of the prior art formulations and methods of the obtaining stabilized and improved recombinant alpha-fetoprotein (rAFP), or its fragments (domain thereof) conjugated PLGA (PLGA-COOH) nanoparticles carrying the various anti-tumor active pharmaceutical substances for the effective therapy of cancer and autoimmune disease; and consequently it is clear that there is a need in the art for an improvement to existing formulations and methods of producing and using rAFP. In this regard the instant invention substantially fulfills these needs.

### SUMMARY OF THE INVENTION

In view of the foregoing disadvantages inherent in the known types of compositions and methods of producing rAFP now present in the prior art, the present invention provides a highly stable rAFP composition and method of producing and using wherein the same can be utilized for producing a high yield of rAFP and increasing the purity of biologically active rAFP to create cytostatic drugs.

It is therefore an object of the present invention to provide a new and improved rAFP and method of producing and using that has all of the advantages of the prior art and none of the disadvantages.

It is another object of the present invention to provide a new and improved method of producing rAFP that significantly increases the yield of pure rAFP that can be used to create conjugates and cytokines for treatment of cancer and autoimmune diseases.

Another object of the present invention is to provide a new and improved method of producing rAFP that provides an effective single or multi-stage purification process that results in pure forms of rAFP and its fragments, wherein the rAFP and its fragments may be used to create pharmaceutically active conjugates.

Yet another object of the present invention is to provide a new and improved method of producing and using rAFP that fulfills the Current Good Manufacturing Practice (CGMP) regulations to ensure quality, potency, and purity of rAFP and its fragments or analogs in conjugates of anti-cancer substances.

Still yet another object of the present invention is to provide a new and improved rAFP in a liquid and dry lyophilized or solid form.

Still yet another object of the present invention is to provide a new and improved method of producing rAFP that includes the steps of introducing rAFP into a methylotrophic strain of Pichia pastoris yeast, allowing the yeast to replicate, and extracting the newly created rAFP.

Still yet another object of the present invention is to provide a new and improved method of using rAFP that includes the steps of combining newly created rAFP with dactinomycin to form cytostatic drugs.

Still yet another object of the present invention is to provide a new and improved method of synthesizing a composition of DAC loaded with PLGA conjugated with rAFP to exhibit more beneficial anticancerogenic action without influence on the normal, healthy cells.

Still yet another object of the present invention is to provide a new and improved rAFP and method of producing that eliminates the problems associated with endobacterial insemination of finished drug dosage forms.

Still yet another object of the present invention is to provide a new and improved rAFP that forms pharmaceutically active conjugates that can be used immediately after dissolution for parenteral administration.

Other objects, features and advantages of the present invention will become apparent from the following detailed description.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein the numeral annotations are provided throughout.

FIG. 1A shows survival rates of Hela cervical cancer cells depending upon concentration of dactinomycin.

FIG. 1B shows survival rates of lymphocyte depending upon concentration of dactinomycin.

FIG. 2A shows tumor growth after being treated with dactinomycin incorporated in PLGA.

FIG. 2B shows survival rate of mice after treatment with dactinomycin in bulk compared to dactinomycin incorporated in PLGA and conjugated with rAFP.

### DETAILED DESCRIPTION OF THE INVENTION

For the purposes of presenting a brief and clear description of the present invention, the preferred embodiment will be discussed as used for utilized for increasing the production and purification of biologically active rAFP to form cytostatic drugs.

The present invention utilizes the Pichia pastoris for a heterological expression of the rAFP. Pichia pastoris is a species of methylotrophic yeast that is widely used for protein expression using recombinant DNA techniques. Pichia pastoris is suitable for protein expression because it has a high growth rate and is able to grow on a simple, inexpensive medium. Additionally, Pichia pastoris can grow in either shake flasks or a fermenter, which makes it suitable for both small and large scale production.

The culture medium of yeast includes the concentration of the culture medium, the precipitation of the target protein, and the chromatographic purification using gel filtration and ion exchange chromatography. In the present invention, the strain of yeast Pichia pastoris can be used as a producer of human AFP with the use of plasmid DNA of very high purity and a double-stranded DNA, particularly of the linear or plasmid nature with the content of chromosomal DNA of 0.5% or lower. The DNA is shielded from contaminating components, such as fragmented chromosomal DNA, endotoxins, proteins, nucleases, and the like. The concentration of culture medium is carried out using cross-selective purification membranes of low-molecular fragments. The culture medium is prepared from the appropriate amount of the following solutions: fermentation basal salts; PTMi trace salts; approximately 75 ml per liter of initial fermentation volume of 50% glycerol containing 12 mM PTMi trace salts per liter of glycerol; approximately 740 ml per liter initial fermentation volume of 100% methanol containing 12 ml PTMi trace salts per liter of methanol.

The list of ingredients for the fermentation basal salts is as follows: 26.7 ml phosphoric acid, 85%; 0.93 g calcium sulfate; 18.2 g potassium sulfate; 14.9 g magnesium sulfate heptahydrate; 4.13 g potassium hydroxide; 40.0 glycerol; and 1 L water. The foregoing ingredients were added to fermenter with water to the appropriate volume and sterilized. The list of ingredients for the PTMi trace salts is as follows: 6.0 g cupric sulfate pentahydrate; 0.08 g sodium iodide; 3.0 g manganese sulfate dihydrate; 0.2 g sodium molybdate dihydrate; 0.02 g boric acid; 0.5 g cobalt chloride; 20.0 g zinc chloride; 65.0 g ferrous sulfate heptahydrate; 5.0 ml sulfuric acid; and water to a final volume of 1 L. The final solution was sterilized and stored at room temperature, or approximately 20 to 25 degrees Celsius.

During the cultivation process, rAFP is introduced into a strain of Pischia pastoris and the yeast is replicated with the use of traditional biochemical methods. The GS115 (his4) strain of the methylotrophic yeast Pichia pastoris is replicated using the methods of replica-plating, as recommended by the supplier (Invitrogen Corp.). It is preferred that the minimum time of cultivation is approximately 24 hours. Additionally, the cultivation process is carried out at approximately −23 degrees Celsius. The lower cultivation temperature helps improve the product yield of human rAFP, which attributes to reduced protease levels, improved folding, as well as reduced toxicity of the immunotoxin to Pichia.

Thereafter, newly created rAFP is extracted from the yeast medium. To separate and purify the newly created rAFP, ethanol/K₂HPO₄ aqueous two-phase system (ATPS) and hydrophobic interaction chromatography are provided from high density fermentation broth. The extraction of rAFP from the fermentation broth attained an average recovery of 100.4%. At the same time, 99.8% of cells and 87.2% of polysaccharides, as well as some other protein impurities are also removed. Because Pischia pastoris lacks the Golgi-resident α-1,3-mannosyltransferase, the newly created rAFP possesses strong antigenic determinants, which enables the obtaining of secretory authentic forms of recombinant proteins.

The extracted rAFP is further purified by removing the yeast cells via centrifugation at approximately 3,000 g for 15 minutes at 4 degrees Celsius. All subsequent operations are then carried out at temperature of 4 degrees Celsius because it is the optimal temperature to support the native structure of proteins. The supernatant liquid is passed through a nitrocellulose filter with a pore diameter of approximately 0.45 microns to separate a filtrate. The filtrate is concentrated on an ultrafiltration device using membranes that cut off the supramolecular structures with weight less than 50 kDa.

Next, low molecular weight components of the environment are removed, thereby reducing the original volume to 100-200 ml. To remove the low molecular weight substances from solution of rAFP, one or more of the following methods that are known in the art have been used, depending upon embodiment: a) ultrafilteration in the membrane having a molecular weight exclusive limit of approximately 1,000 to 50,000 preferably from about 10,000 to about 30,000; b) cation exchanger treatment for functional groups such as sulfo groups, carboxyl groups, and the like; c) hydrophobic chromatography treatment; d) anion exchanger treatment; and e) salt precipitation.

Salt precipitation of the target protein is then performed using a 60% solution of ammonium sulfate. Precipitate of the target protein is collected by centrifugation at approximately 20,000 g for 20 minutes. The obtained precipitate is dissolved in 50 mM ammonium acetate buffer (pH 5.5) containing 10 mM inosine and 0.05% polysorbate-20. In the present invention, commercially available Tween-20 (polysorbate-20) was used. The use of 0.05% Tween-20 increases stability to the composition of the drug on the basis of rAFP. Once dissolved, gel filtration is performed. The precipitate is applied on a column containing 1,000 ml Sephacryl S 1000, balanced with 50 mM ammonium acetate buffer (pH 5.5) containing 10 mM inosine and 0.5% Tween-20. The initial volume of 350 ml is discarded to help decrease the impurity of the newly formed rAFP, and the remaining volume containing the target protein are pooled as eluate.

The eluate is titrated with 10% ammonia to pH 7.0 and applied on a column containing 50 ml of diethylaminoethyl cellulos (DEAE-C), then equilibrated with 50 mM ammonium acetate buffer (pH 7.0) containing 10 mM inosine and 0.05% Tween-20. The column is washed successively with 100 ml of equilibrating buffer and 100 ml of equilibrating buffer containing 0.1 M sodium chloride. The two-step chromatographic purification of rAFP in the presence of 0.5% Tween-20 and 10 mM inosine prevents aggregation of the monomers of the target protein, thereby increasing the yield of biologically active rAFP. Elution is then performed using a gradient of sodium chloride concentration from 0.1-1.0 M in 50 mM ammonium acetate buffer (pH 5.5) containing 10 mM inosine and 0.05% Tween-20 with subsequent clarification of the protein fraction by centrifugation at 100,000 g.

Electrophoresis in reducing conditions show that the molecular weight of obtained proteins is approximately 20 kDa. The purified rAFP appears as a single band on reduced SDS-PAGE gel, and it has a purity of 98.9% as determined by HPLC. In using the present method of purification through the foregoing single or multistage purification processes, the impurity content of rAFP does not exceed 0.5%. The specific activity is approximately 0.3-1.0×108 IU/mg. Average yield is approximately 7-9 mg, or approximately 78.7% from one liter of the culture medium.

The biologically active rAFP can be used immediately after dissolution for parenteral administration. The present invention provides a stable pharmaceutical preparation based on rAFP or its fragments in complexes or conjugates. The rAFP may be used in the prevention or treatment of oncological and autoimmune-diseases, as well as for the target therapy of cancer. Without limitation, the newly formed rAFP of the present invention may be used with cytostatic drugs such as dactinomycin D, cardionolide, bufadinolide, inhibitors of tyrosine and others kinases, inhibitors of Hedgehog and mTOR signaling pathways, cardiotonic steroids (e.g., oleadrin, digoxin, digitoxin), anti-rheumatic substances (e.g., methotrexate, cyclophosphamide, sulfasalazine), or inhibitors of proteasomes.

In one embodiment, DAC-loaded PLGA nanoparticles may be prepared using a modified simultaneous double-emulsion, or water-in-oil-in-water (W/O/W) solvent evaporation and diffusion technique. 100 mg PLGA-COON is dissolved in 10 ml organic mixture of water immiscible organic solvent dichloromethane (DSM) and water partial miscible solvent acetone (ACE) with various ratios (from 5:0 to 2:1, v/v) containing Tween-80 (5%, v/v) as emulsifier. DAC is dissolved in 2 ml of distilled water and then emulsified in the polymer solution through homogenization for approximately five minutes, and repeated as necessary. The primary water in oil (W/O) emulsion is further added to 40 ml of external water with homogenization for three minutes to achieve the stable double emulsion.

The resulting emulsion is dropped gradually into a 200 ml of aqueous solution with different surfactants such as Pluronic F68, polyvinylalcohol (PVA), and gelatin, under steady stirring to make the nanoparticles solidify. The residual organic solvents is evaporated under negative pressure. Thereafter, nanoparticles suspending in emulsion is collected by ultracentrifugation at 12000 rpm and washed with distilled water three times. Finally, the suspension is then lyophilized for two days at approximately −41 degrees Celsius and no more than 10 μm mercury pressure to obtain powered nanoparticles. The powdered PLGA nanoparticles may be stored at 4 degrees Celsius.

DAC-loaded PLGA nanoparticles are conjugated with rAFP through a carbodiimide coupling reaction. For this purpose, PLGA carboxylic functional groups are first activated through exposure with 15 mg 1-ethyl-3-(3-dimethylaminopropyl) carbodiimide (EDC) and 12 mg N-hydroxysuccinimide (NHS) for approximately five hours, preferably following their preparation in a medium consisting of buffering solution having a pH level of 8.0. The activated nanoparticles are then purified by three times centrifugation at 21,000 g for thirty minutes at 10 degrees Celsius to remove any remnants of PVA, EDC and NHS. The activated DAC-loaded PLGA nanoparticles are then exposed to rAFP solution in 40 ml phosphate buffered saline (PBS) having a pH level of 7.4 with an equimolar concentration in a dropwise manner and under constant stirring. The conjugated rAFP-DAC-loaded PLGA nanoparticles are then isolated by centrifugation at 15,000 g for thirty minutes at 10 degrees Celsius and the supernatant is removed. Conjugated rAFP-DAC-loaded PLGA composition is then subjected to the lyophilization process for long term storage.

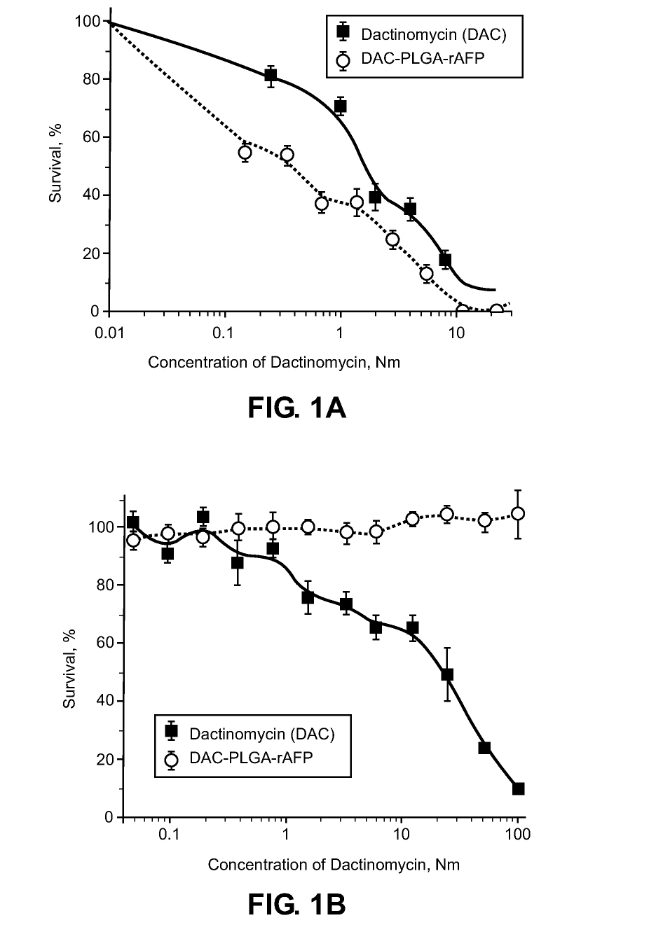

The cytotoxicity of dactinomycin incorporated in PLGA conjugated with rAFP was tested with Hela cervical cancer cells. It is recognized that the use of PLGA as the controlled release polymer system is particularly suitable for the intent of the present invention because PLGA is well known for its safety in clinic. The Hela cells were doused with both dactinomycin in bulk. MTT assay was then performed to quantify the inhibitory concentration. As shown in FIG. 1A, dose-dependent survival of Hela cervical cancer cells under the influence of dactinomycin in bulk (1.7 nM) as compared to dactinomycin incorporated in PLGA and conjugated with rAFP (0.4 nM) shows 50% growth inhibition concentration (1050). 1050 was calculated from the cell viability data as the drug concentration in which cell growth was inhibited by 50%.

Similarly, as shown in FIG. 1B, dose-dependent survival of lymphocyte of an average healthy person under influence of the dactinomycin in bulk (23.8 nM) as compared to dactinomycin incorporated in PLGA and conjugated with rAFP (>100 nM) shows 50% growth inhibition concentration. Each value depicted in FIGS. 1A and 1B is the mean of three determinations with coefficient of variation less than 20%.

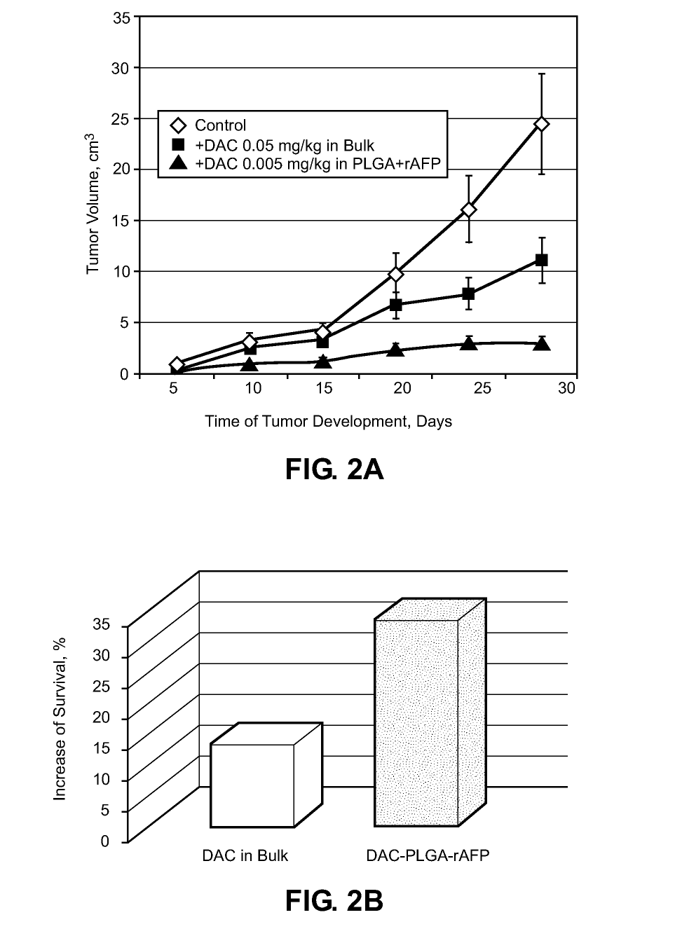

FIGS. 2A and 2B depict an example of antitumor activity of dactinomycin incorporated in PLGA, wherein dactinomycin is in vivo in the experimental model of P388 murine leukemia cells in six to eight week old male DBA/2 mice. 1.0×10⁶ P388 murine leukemia cells were injected into the peritoneal cavity of the mice. The P388 murine leukemia cells were maintained in vitro as a suspension culture in RPMI1640 medium supplemented with 10% fetal calf serum, 100 μg/ml penicillin and 50 μg/ml. Chemotherapy was initiated twenty four hours after the introduction of tumor cells. Thereafter, one group of mice were treated with dactinomycin in bulk, DAC 0.05 mg/kg per dose, once a day for three days. Similarly, another group of mice were treated with dactinomycin that was incorporated in PLGA and then conjugated with rAFP, DAC 0.005 mg/kg in PLGA+rAFP per dose, once a day for three days.

As shown in FIG. 2A, the tumor volume is significantly decreased over elapsed time when dactinomycin is incorporated in PLGA and conjugated with rAFP, as compared to dactinomycin in bulk. Each experiment was performed in duplicate to verify the reproducibility of the results. Comparing the results obtained from the present cytotoxic treatment of the first and second group of mice, the use of dactinomycin incorporated in PLGA and conjugated with rAFP increases the likelihood of survival by at least 100%, as shown in FIG. 2B. Additionally, the composition of DAC loaded with PLGA conjugated with rAFP exhibited more beneficial anticancerogenic action without influence on the normal, healthy cells.

In one embodiment, the pharmaceutical composition of the drug in a dry lyophilized form contains the following ingredients: 10-100 mcg/ml rAFP; 5-40 mg/ml inosine; 0.5-5 mg/ml Tween; 10-20 mM Buffer solution (pH 5.5). The rAFP in a dry lyophilized form, or solid form, may be used as tablets, capsules, powders, granules, or suppositories. Additionally, solid forms of rAFP may be suitable for external use, such as creams, gels, or liquid preparations. These are obtained by dissolving the solid form in a pharmaceutically acceptable solvent obtained by standard technological means.

In another embodiment, the pharmaceutical composition of the drug in a liquid and dry lyophilized form also contain the following ingredients: 10-100 mcg/ml rAFP; 5-40 mg/ml inosine; 0.5-5 mg/ml Tween; 10-20 mM Buffer solution (pH 5.5). The rAFP in a liquid form, may be used as emulsions, syrups, elixirs, drops, liposome forms, or nanoforms. Additionally, liquid forms of rAFP may be suitable for external use, such as gels and wet wipes.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A method for increasing production and purification of rAFP, comprising the steps of:
introducing rAFP into a methylotrophic strain of Pichia pastoris yeast;
allowing said yeast to replicate;
extracting newly created rAFP;
precipitating said newly created rAFP using an ammonium sulfate solution to form a precipitate;
purifying said precipitate using a chromatographic purification process in the presence of an inosine solution and a polysorbate-20 solution;
conducting gel filtration in a first ammonium acetate buffer containing said inosine solution and said polysorbate-20 solution;
conducting ion-exchange chromatography on DEAE-C;
conducting elution of the target protein by gradient of concentration of a sodium chloride solution in said first ammonium sulfate buffer containing said inosine solution and said polysorbate-20 solution.

2) The method for increasing production and purification of rAFP of claim 1, wherein a concentration of said culture medium is carried out using cross-selective purification membranes of low-molecular fragments.

3) The method for increasing production and purification of rAFP of claim 1, wherein said precipitate comprises said newly created rAFP.

4) The method for increasing production and purification of rAFP of claim 1, wherein said chromatographic purification process comprises:
dissolution of said precipitate in said first ammonium acetate buffer containing said inosine solution and said polysorbate-20 solution.

5) The method for increasing production and purification of rAFP of claim 1, wherein said first ammonium acetate buffer has a pH level of 5.5 and a molarity of 50 mM.

6) The method for increasing production and purification of rAFP of claim 1, wherein ion-exchange chromatography on DEAE-C uses a second ammonium acetate buffer as a balancing solution containing said inosine solution and said polysorbate-20 solution

7) The method for increasing production and purification of rAFP of claim 6, wherein said second ammonium acetate buffer has a pH level of 7.0 and a molarity of 50 mM.

8) The method for increasing production and purification of rAFP of claim 1, wherein said ammonium sulfate solution is 60% solution.

9) The method for increasing production and purification of rAFP of claim 1, wherein a molarity of sodium chloride solution is between 0.1 M and 1.0 M.

10) The method for increasing production and purification of rAFP of claim 1, wherein a molarity of said inosine solution is 10 mM.

11) The method for increasing production and purification of rAFP of claim 1, wherein said polysorbate-20 solution is 0.5% solution.

12) A method of treating an autoimmune disease in a mammal, comprising the steps of administering to said mammal a therapeutically effective amount of dactinomycin incorporated in PLGA and conjugated with rAFP.

13) The method of treating an autoimmune disease of claim 13, wherein said mammal is a human patient.

14) A cancer and autoimmune disease treatment composition, comprising rAFP conjugated through a carbodiimide conjugation reaction with active substances incorporated in PLGA.

15) The composition of claim 14, wherein said active substances comprise a dactinomycin.

16) The composition of claim 14, wherein said active substances comprise inhibitors of kinases.

17) The composition of claim 14, wherein said active substances comprise cardiotonic steroids.

18) The composition of claim 14, wherein said active substances comprise anti-rheumatic substances.

19) The composition of claim 14, wherein said active substances comprise inhibitors of proteasomes.

20) The composition of claim 14, wherein said active substances comprise inhibitors of Hedgehog and mTOR signaling pathways.
